(19) United States
(12) Patent Application Publication
(10) Pub. No.: US 2015/0038892 A1
(43) Pub. Date: Feb. 5, 2015

(54) PATCH STRUCTURES FOR CONTROLLED WOUND HEALING

(71) Applicant: ETH ZURICH, Zurich (CH)

(72) Inventors: Anastasios Marmaras, Zurich (CH); Vartan Kurtcuoglu, Winterthur (CH); Aldo Ferrari, Zurich (CH); Dimos Poulikakos, Zollikon (CH)

(73) Assignee: ETH ZURICH, Zurich (CH)

(21) Appl. No.: 14/368,201

(22) PCT Filed: Dec. 10, 2012
(86) PCT No.: PCT/EP2012/005097

§ 371 Date: Jun. 23, 2014

(30) Foreign Application Priority Data
Dec. 22, 2011 (EP) 11010105

(51) Int. Cl. A61F 13/02

(52) U.S. Cl. CPC A61F 13/0243; A61F 13/0289; A61F 2013/00157
USPC 602/43; 264/401

(57) ABSTRACT

The invention relates to an active surface element (2) for improved healing of cell layer lesions comprising at least one topographically structured surface on a substrate, with a pattern comprising alternating ridges (5) and grooves (6) with a pattern period (p) and extending along a pattern length (l), wherein the pattern period (p) is smaller than 10 μm and the pattern length (l) is larger than 1 mm. The invention furthermore relates to methods of making such an active surface element as well as to bandages, in particular adhesive bandages comprising such active surface elements.

### TECHNICAL FIELD

The present invention relates to the field of wound healing, in particular to patches for inducing improved wound healing.

### PRIOR ART

Wound dressings are designed to support the wounded region, protect it from infection, and, in certain cases, actively promote wound healing by creating a favorable environment for cell growth.

The response to wounding, defined as a breakage of bodily tissue, involves an inflammation phase, a migratory phase and a remodeling phase. The inflammation phase is the acute response to a wound and its purpose is to quickly seal the wound and produce chemical factors that employ cells to migrate into the wound and start the wound healing process. During the migratory phase, cells rapidly migrate into the wound and start laying down provisional extracellular matrix that will be the base of the healed tissue. During the remodeling stage, the newly created tissue slowly matures into its permanent form.

Standard wound dressings facilitate wound healing by: 1. mechanically holding the wound edges together to allow easier cell migration; 2. mechanically sealing the wound to prevent contamination by pathogens; 3. in some advanced dressings providing an environment that actively promotes faster wound healing, usually by exposing the wounded tissue to a hydrated gel.

### SUMMARY OF THE INVENTION

In currently available wound dressings, there is no exploitation of geometrically controlled mechanical cues to improve the speed and direction of wound healing. This invention encompasses a surface with a microscale pattern that can, when applied to a wound, speed up and improve wound healing through cell contact guidance.

The invention comprises a patch, or active surface element, with microscale patterns to be used as wound dressing. It is for example well suited for large area lesions such as burns or the like, but also for conventional small or large cuts in the skin. It may be applied externally or also internally, so to skin but also to other internal tissue. If applied internally the substrate or backing/carrier material on which the active surface element is mounted (if present) as well as on which the active surface element itself is based, can be biodegradable so that e.g. the wound can be closed above the patch and the patch degrades after having fulfilled its function so as to avoid to have to reopen the wound for removal of the patch. In case of external application normally the active surface element should essentially not be biodegradable, but an optional coating thereon can be biodegradable.

This active surface element exploits a phenomenon known as contact guidance by which migratory cells, when exposed to certain topographical patterns, tend to migrate faster and in an ordered, aligned way. The patterned patch can cause cells to migrate faster into the wound (faster wound healing) and in a more aligned/ordered fashion (lower probability for scarring).

Contact guidance has thus far been associated with a firm yet dynamic link of cells with their extracellular matrix through focal adhesions. With the in vitro experiments given below it is shown that contact guidance can also take place without the involvement of focal adhesions. This is important for the invention, as a firm attachment or adhesion between the wound and the dressing can be avoided allowing for the patch to come off after a given time without re-opening of the wound. This newly observed concept has also positive implications for the choice of the wound dressing material: It does not have to promote cell adhesion, but simply should be biocompatible, which extends the range of usable materials considerably.

What is therefore, among other aspects, completely unexpected is the finding that due to the lack of establishment of focal adhesion between the active surface element and the cells it is possible to use such an active surface element as an external guiding aid for cell growth which can easily and without damage to the newly formed cell layer be removed after the establishment of the new and contiguous cell layer.

The essential new features of this invention are:

a) Enhanced wound healing through contact guidance by microscale patterning of normally 0.5 micrometer or larger depth and normally about 2 micrometer duty cycle. This can be shown to enhance endothelial cell layer growth, but also to enhance wound healing when other cell types are involved

b) Contact guidance without focal adhesion involvement. This is a new biological concept that allows for improved wound healing without the problem of dressing adhesion to the wound types.

Topographic modifications of the substrate have the potential to guide cell polarization and migration, through which they facilitate epidermal wound healing. Classic topographic contact guidance is based on the interaction between cells and a supporting scaffold that interferes with the establishment of focal adhesions, thereby influencing the organization of the actin cytoskeleton. Exploiting soft lithography techniques on PDMS, as detailed below gratings of groove and ridge width of 1 μm and groove depth of 0.6 μm were made. These gratings were applied to the apical, free surface of human dermal fibroblasts during in vitro wound healing. Gratings oriented perpendicularly to the wound induce a significant enhancement of cell polarization, migration speed and directionality which results in faster wound coverage. The apically applied texture influenced the deposition of extracellular matrix into the wound yielding homogenously distributed fibronectin fibers. Apical guidance was not mediated by the establishment of focal adhesions between cells and the topographically modified patch, thus allowing for removal of the latter after complete healing. Altogether, the results below demonstrate an alternative guidance scheme based on the apical, adhesion-free interaction between migrating cells and an anisotropic topography which leads to faster healing in an in vitro wound model.

Indeed, acute mechanical epidermal wounding, defined as the breaking of continuity in an epidermal tissue, is followed by a wound healing response organized in three basic stages: Inflammation, tissue formation and tissue remodeling. During tissue formation, the wound is populated by cells mostly through directional migration from the wound edges. In particular, dermal fibroblasts rapidly migrate into the wound where they become the dominant cell population and produce an early provisional extracellular matrix (ECM) mainly consisting of newly deposited collagen and fibronectin.

Whether and to what extent the healing process results in new functional tissue or in a scar critically depends on the proper and fast execution of all wound healing stages. Fibroblasts play a central role during the initial phase of tissue formation; their migration constitutes a rate-limiting step that controls the outcome of the subsequent processes. Indeed, a slow and inefficient wound colonization by fibroblasts results in scar formation. Additionally, the architecture of the ECM deposited by fibroblasts into the wound area governs the ensuing migration of epidermal cells. In particular, inhomogeneous distribution of ECM fibers is linked to scarring.

During migration, fibroblasts follow a number of overlapping directional signals that derive from gradients of soluble molecules as well as from the chemical and physical properties of the extracellular environment. In particular, the local topography of the ECM influences cell polarization and migration in a process termed contact guidance.

Contact guidance requires signal transmission through transmembrane integrin receptors that directly recognize and bind to specific epitopes in the ECM. Integrin engagement fosters the establishment and maturation of a cytoplasmic complex, the adhesion plaque, which in turn provides the functions of signal transduction and mechanical anchoring between the cell and the substrate. Initial small integrin-based adhesions enlarge and mature into larger focal adhesions by recruiting a number of adaptor, signaling or actin-regulator proteins to the adhesion site. Mature focal adhesions eventually connect with the actin cytoskeleton through proteins such as vinculin. In this way the adhesions to the substrate can remodel the cell shape and polarization during migration.

Topographical modifications of surfaces such as grooves deeply influence the polarization and migration behaviour of several cell types including neurons, epithelial cells, and fibroblasts. These scaffolds mimic the interaction between cells and ECM, imposing geometrical constraints to the establishment and maturation of focal adhesions. Fibroblasts, in particular, readily respond to gratings with lateral feature size between 0.1 μm and 10 μm by polarizing and migrating along the topography. The deposition and remodeling of ECM fibers by migrating fibroblasts is similarly influenced by topographically-modified substrates.

One common characteristic of contact guidance studies is that cells are forced to assemble integrin-based adhesions at the interface with the structured surface. Hence, upon wound healing, the artificial scaffold is integrated into the regenerated tissue and cannot be removed. This is in contrast with the application of a textured substrate on the free surface (i.e. the apical cell surface) of an existent cell layer, which more closely resembles the deployment of an engineered dressing on a wounded epidermis. The possibility of guiding cell migration through the interaction with the apical cell surface has not been investigated until now.

Below it is shown that it is indeed possible to influence fibroblast migration and polarization as well as the architecture of newly deposited ECM through an apically applied topography. Additionally, the data imply that this guidance effect is obtained without the establishment of focal adhesions between migrating cells and the textured surface, thus allowing for clean removal of the wound dressing after wound closure. A novel ‘top guidance’ mechanism that opens the door to new approaches for the support of epidermal wound healing is thus presented.

More specifically, the present invention relates to an active surface element for improved healing of cell layer lesions comprising at least one topographically structured surface on a substrate, with a pattern comprising alternating ridges and grooves with a pattern period p and extending along a pattern length l, wherein the pattern period p is smaller than 10 μm [micrometer] and the pattern length l is larger than 1 mm. For a schematic definition of these parameters reference is made to FIG. 1 and FIG. 2a for the case of a rectangular ridge/groove structure.

The pattern length (length dimension perpendicular to the illustration given in FIG. 2a) should be >1 mm. Alternatively or in addition to that for most applications the pattern length l should be larger than a multiple of the feature's size, so for example it should be more than 20 times, preferably more than 100 times the period p of the structure.

According to a preferred embodiment of the invention, the width of the ridges and/or of the grooves is in the range of 1-9 μm [micrometer]. More preferably the width of the ridges is in the range of 1-5 μm [micrometer] and the width of the grooves is in the range of 1-5 μm [micrometer], preferably both widths being essentially equal. So normally ridge or groove width is between 1-9 μm or 2-9 μm.

According to yet another preferred embodiment the ridges have a height h of at least 0.5 μm [micrometer], preferably in the range of 0.5-5 μm [micrometer], more preferably in the range of 0.5-2 μm or 1-2 μm [micrometer]. So normally the pattern height is between 0.5-2 μm, while the upper limit is generally not biologically important, it's normally only to make sure that the device is rigid, more below.

The sidewalls of the grooves and a bottom wall of the grooves (in case of a flat bottom wall) enclose a pattern angle α in the range of 85-120°, wherein preferably the pattern angle (α) is around 90°. There is not necessarily a flat bottom wall of the grooves, is also possible that the pattern is a sequence of triangular ridges with sidewalls contacting each other on the bottom of the ridge, in this case the angle enclosed by the two sidewalls of the grooves is typically in the range of 30-90°. In case of such triangular structures the correspondingly formed ridges can have a flat top, a rounded top or an edge forming top as illustrated in FIG. 2c.

The ridges and/or the grooves are for example mirror symmetric with respect to a respective central plane arranged essentially parallel to the running direction of the pattern. According to yet another preferred embodiment the surface element comprises a substrate based on or consisting of a biocompatible polymeric material, preferably selected from the group consisting of: polycaprolactone, polyethylene glycol, polylactic acid, polyglycolic acid, polybutyric acid, as well as mixtures, derivatives, hydrogels and copolymers thereof. The substrate or a coating on the surface of the substrate may further comprise particular, wound healing assisting and/or inflammation preventing substances and/or pharmaceuticals incorporated correspondingly suitable amounts.

The patch should have, unless there is a strong carrying structure as a backing, a sufficiently rigid self-supporting structure, and the inherent rigidity should also make sure that there is no deformation upon application to the lesion impairing that the topographical structure and consequentially its influence on cell migration. Correspondingly therefore the biocompatible polymeric material preferably has a Young's modulus of at least 100 kPa, preferably in the range of 100 kPa-10 GPa.

The surface can be coated, uncoated and/or plasma treated. If coated such a coating can be a monolayer coating and it may comprise wound healing assisting and/or inflammation preventing substances and/or pharmaceuticals. It is normally biodegradable, and preferably biodegradable on a shorter time scale than the substrate of the patch.

The substrate for many applications may have an open (or effective) porosity, preferably with pores with a diameter in the range of 1μ-1 mm, preferably in the range of 1μ-2μ.

The effective porosity (also called open porosity) refers to the fraction of the total volume in which fluid flow is effectively taking place and includes Caternary and dead-end pores and excludes closed pores (or non-connected cavities). This is important for solute transport. This however does not exclude that there is additional) closed porosity, this is however not contributing to exchange of steam and/or liquid and/or air from the lesion to the outside.

The surface elements may further include a backing material adhesively (or otherwise) attached on the side opposite to the topographically structured surface, wherein said backing material is normally adapted for supporting the surface element and/or for allowing to adhesively attach the combined structure to the skin of a patient. Preferably the backing material is a multilayer structure including for example layers for absorption as well as layers for adhesion purposes.

The backing material can be an absorbent backing material, preferably selected from the group consisting of: cotton, viscose, cellulose, silk, or combinations thereof, in woven or nonwoven forms.

Furthermore the present invention relates to a method for making a surface element as outlined above, wherein a topographically complementary structured mould element is used as a template for a liquid applied or injected substrate material, preferably in a soft lithography process, optionally followed by a cross-linking and/or polymerization step, further optionally followed by a surface treatment step, preferably a plasma treatment step on the topographical surface.

Last but not least the present invention relates to a bandage, preferably an adhesive bandage comprising at least one surface element as outlined above, wherein preferably the orientation of the pattern length l of the surface element on the adhesive bandage is arranged such as to lie essentially perpendicular to the corresponding lesion, preferably to a skin lesion, or preferably including a cut in epidermis and/or dermis and/or hypodermis cell layers.

In addition to that, the invention relates to a method for wound healing of cell layer lesions, preferably skin cell layer lesions, most preferably lesions in epidermis and/or dermis and/or hypodermis cell layers, comprising the step of applying a surface element as outlined above on the lesion, preferably in a relative orientation such that the orientation of the pattern length l is under an acute angle or preferably essentially perpendicular to the main orientation of the lesion (so e.g. the direction of the cut), allowing the regeneration of the cell layers, and removal of the surface element or biodegradation of the surface element.

Further embodiments of the invention are laid down in the dependent claims.

The results below demonstrate the possibility of guiding the migration of human dermal fibroblasts through the application of a micro-engineered patch on the apical, free surface of a wounded cell monolayer. The interaction between the featured anisotropic topography and the migrating cells is sufficient to enhance cell polarization and favor directional migration into the wound, thereby promoting wound coverage. This ‘top guidance’ process is further reflected in the architecture of the extracellular matrix, which is newly deposited on the basal support within the wounded region. Importantly, this set of guidance effects is obtained without the establishment of integrin-based adhesions between the cell and the apical patch, thus allowing patch removal after healing without damaging the cell layer. Contact guidance by textured basal substrates can be described as a bottom-up process mediated by the biological interaction between the cell and the underlying topography. In this scenario, topographical features of various sizes and shapes act as physical barriers that hamper or hinder the establishment and maturation of integrin-based adhesions. This interaction eventually results in a geometrical constraint of focal adhesion maturation such that, when the substrate topography supporting the cell is anisotropic, the majority of focal adhesions is established and matures along the direction dictated by the substrate. The distribution of adhesions is then linked to the overall remodeling of the cell shape by the assembly of actin stress fibers and by the generation of cell-mediated contractility. With the same mechanism, migrating cells are restricted on their path by the topographical boundaries provided by the substrate.

In contrast, ‘top guidance’ is not mediated by integrin-based adhesions. Under our experimental conditions, although exposed to two chemically identical interfaces, fibroblasts maintained an unaltered apico-basal polarity. Indeed, they conserve focal adhesions and deposit fibronectin on the lower, unstructured, basal support. Without being bound to any theoretical explanation it appears that the effect of the apically-applied topography results from an anisotropic shear stress distribution on the apical cell surface:

Movements parallel to the gratings may therefore minimize the mechanical resistance in migrating cells. In this way the polarization of cell activities induced by ‘top guidance’ may work in synergy with the inherent wound healing stimulus resulting from the loss of cell-cell contacts upon wounding. Indeed, cell confinement in narrow PDMS channels or within parallel layers of agarose has been shown to induce friction-based motility in cancer and immune cells. The observation that cells can still migrate when the apical gratings are oriented parallel to the wound (and thus perpendicularly to the healing direction) supports the hypothesis that ‘top guidance’ originates from a restriction of cell movements that can be overcome by the signals driving fibroblasts into the wound.

Importantly, the interaction between apical topography and migrating fibroblasts is transmitted to the basal cell surface: Cells display an overall stronger migration phenotype characterized by an increased number of immature adhesions and by a stronger orientation of the actin cytoskeleton. Additionally, ‘top guidance’ affects the basal deposition of fibronectin fibers into the wound area. The conversion of apical shear stress distribution into a global adaptation of the cell shape and activities requires a mechanical transduction which may be provided by a cortical actin structure or through the deformation of the cell nucleus.

Finally, the reported findings open the door to new approaches in tissue engineering and wound care. The possibility to control cell migration and matrix deposition without triggering a biological interaction with the underlying tissue yields removable wound dressings that improve wound healing and reduce scar tissue formation.

### BRIEF DESCRIPTION OF THE DRAWINGS

Preferred embodiments of the invention are described in the following with reference to the drawings, which are for the purpose of illustrating the present preferred embodiments of the invention and not for the purpose of limiting the same. In the drawings,

FIG. 1 shows a schematic healing patch, illustrating important dimensions, wherein the active surface is composed of alternating lines of grooves and ridges;

FIG. 2 shows a cut essentially perpendicular to the running direction of the grooves/ridges with the possible dimensions schematically illustrated in a), and in b)-d) possible alternative shapes of the grooves/ridges;

FIG. 3 shows an illustration of the experimental setup, wherein in (A) A PDMS active surface element or patch is generated by soft lithography, in (B) the PDMS patch is plasma-treated to obtain a hydrophilic surface for supporting the gelatin coating (green), in (C) a confluent layer of primary human dermal fibroblasts (HDF) is obtained by culturing cells on a gelatin coated basal support (a Petri dish) in (D) the monolayer is mechanically wounded and in (E) the active surface of the patch is applied over the wound;

FIG. 4 shows the enhanced wound coverage through apical application of perpendicularly oriented gratings, wherein (A) shows fluorescent images extracted from a time-lapse of fibroblast wound healing under the PDMS apical patch, the orientation of the gratings is shown in the last panel (t=24 h); (B) corresponding fluorescent images extracted from a control time-lapse of wound healing under a blank patch, the entire wounded region is visible at time 0 h (left panel), a white rectangle at time 0 h defines a region of interest in the wound, a zoomed view of the corresponding region of interest is reported at time 12 h (middle panel) and 24 h (right panel); (C) comparison of wound healing dynamics under perpendicular gratings (gray curve) or blank patch (black curve); and (D) comparison of wound healing dynamics with parallel gratings (gray curve) or blank patch (black curve), the graph insets display the statistical significance (p-value) at each time of measure;

FIG. 5 shows the apical guidance of fibroblast migration, wherein in (A) characteristic tracks of individual cells migrating into the wound under perpendicular gratings, or (B) blank patch; (C) a comparison of individual cell displacement, average velocity, and migration directionality upon wound healing under perpendicular gratings (gray) or blank patch (black; p<0.001); a (D) distribution of individual track orientation (relative to the blank control) for cells migrating into the wound under perpendicular gratings, an orientation of 90° indicates alignment perpendicular to the wound;

FIG. 6 shows cell polarization along the gratings, wherein (A) orientation of cells migrating into the wound under perpendicular gratings (gray) or blank patch (black), the orientation of randomly migrating cells in subconfluent cultures (light gray) is reported as control (gratings vs. blank: p<0.001, gratings vs. control p<0.001); (B) orientation of actin microfilaments and focal adhesions in cells migrating under perpendicular gratings (B) or a blank patch (C); the pictures report the inverted fluorescent signal at the as revealed by LifeAct-EGFP (top panel) and Vinculin-FRP (middle panel) expression, respectively. The bottom panel shows an overlay of the green (LifeAct-GFP) and red (Vinculin-FRP) fluorescent channels;

FIG. 7 shows the architecture of cell-deposited fibronectin, wherein the apical interaction with perpendicular gratings influences the deposition of fibronectin by migrating fibroblasts is shown, and wherein in (A) an inverted fluorescent image of fibronectin fibers deposited on the basal support by cells migrating in the wound area under the perpendicular gratings, or (B) blank patch are shown, in (C) randomly oriented fibronectin deposited by cells in unwounded regions, in (D) orientation of fibronectin fibers deposited in the wound region under perpendicular gratings (gray), blank patch (black) or in an unwounded region (light gray), an orientation of 90° indicates alignment perpendicular to the wound (gratings vs. blank: p=0.02, gratings vs. control p=0.02), in (E) homogeneity of the fibronectin matrix (gratings vs. blank: p=0.005, gratings vs. control p=0.004). Coloring as in (D);

FIG. 8 shows the focal adhesions establishment and maturation by migrating fibroblasts, wherein in (A) focal adhesions established at the interface between the cell and the basal support are shown, in (B) comparison of the number of focal adhesions established at the interface with the basal support or the apical patch, under the perpendicular gratings and blank patches (p=0.04), in (C) comparison of focal adhesion size of cells under the patterned and blank patches (p=0.004);

FIG. 9 shows the water static contact angle measured on the active PDMS patch surface upon different plasma treatments, the contact angle of untreated PDMS patches is compared with the contact angle of patches treated with low power (10 W) plasma for 30, 60, 90, 120, and 150 seconds and with the contact angle of gelatin coated PDMS;

FIG. 10 shows the image processing and analysis, wherein in (A) the raw fluorescent image is shown, in (B) the contrast enhanced fluorescent image, in (C) the thresholded image, in (D) the automatic wound boundary detection, in (E) the calculation of cell coverage of the wounded region during a wound healing experiment, and in (F) individual cell detection and alignment calculation; and

FIG. 11 shows the patch removal after complete wound healing, wherein in (A) an illustration of a wounded monolayer before and (B) after patch application is shown, in (C) the healed monolayer before and (D) after patch removal, and in (E) a DIC image of a healed region before and (F) after patch removal.

### DESCRIPTION OF PREFERRED EMBODIMENTS

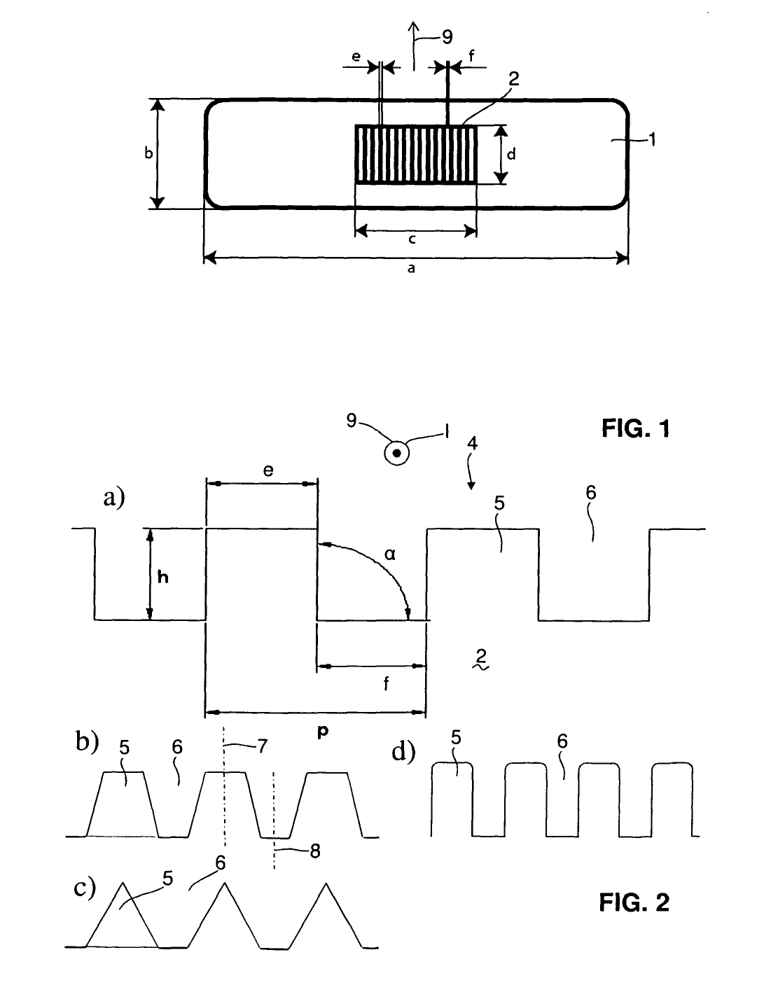

FIG. 1 shows, schematically, an adhesive bandage or healing patch 1 with an active surface element 2. The healing patch comprises a large, conventional strip of bandage material, the backing material, which is normally at least partially transmissive for air, humidity and/or liquids, however it can also be a scaling material without transmissive properties. At least in certain regions it is normally provided with a layer of pressure sensitive adhesive (on the side facing the viewer in FIG. 1), which prior to use may be covered by a covering layer which is removed prior to the application of the adhesive bandage.

Behind the active surface element 2, so between the active surface element 2 and the backing material there may be provided additional absorbing layers, for example gauze layers.

The active surface element 2 is arranged such that the running direction 9 of the micro-pattern of the active surface element 2 is arranged perpendicularly to the typical scar direction to be covered by the adhesive bandage. In this case the scar direction is typically arranged essentially parallel to the long axis of the adhesive bandage with a length a which is larger than the width b. Typically the length c of the patterned active surface element 2 is smaller than the length a of the adhesive bandage backing material, and the width d of the patterned active surface element 2 is also smaller than the width b of the adhesive bandage backing material strip.

The active surface element 2 has grooves 6 with a width f and ridges 5 with a width e. This shall be illustrated in somewhat more detail in the context of FIG. 2, specifically FIG. 2a, in which a cut essentially perpendicular to the running direction of the pattern on the active surface element 2, so perpendicular to the arrow 9 in FIG. 1, is shown. In this case the pattern is a regular rectangular pattern, where both widths e and f are equal, and where the pattern angle α is 90°. The length l of the actual pattern needs to have the minimum length as outlined above, and normally this length l is equal to the full with d of the active surface element 2 as illustrated in FIG. 1. The ridges have a height h (or the grooves have a depth), which can be within the boundaries as outlined above.

The shape of the pattern does not need to be a regular rectangular shape as illustrated in FIG. 2a. The ridges can also be of at least partly trapezoidal shape as illustrated in FIG. 2b, they can be of triangular shape as illustrated in FIG. 2c (it is also possible that the triangles meet at the bottom of the ridges leading to a zigzag shape), and they can also be rectangular with rounded edges as illustrated in FIG. 2d (the rounded edges can be at the top corners of the ridges as illustrated in FIG. 2d, they may however also be or alternatively be at the bottom edges of the grooves).

Within FIGS. 1 and 2 only situations are shown where the pattern essentially extends along a single linear direction. It is however also possible to have a bent structure along the direction 9, if growth of the cells is to be induced along such a bend. The length l with the limits as outlined above is in this situation to be understood as the length along such a bent shape.

Patch Fabrication:

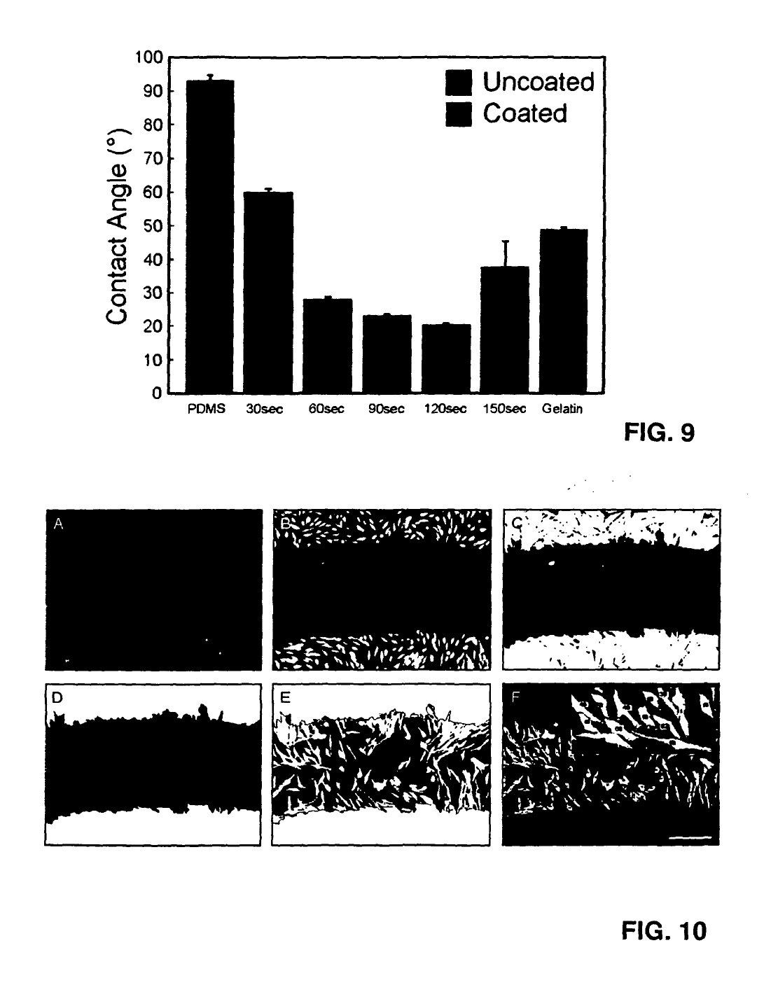

Patches to assist wound healing were made of Polydimethylsiloxane (PDMS, Dow Corning, USA) at 1:10 mixing ratio. The mixed PDMS was degassed in a vacuum chamber for 10 minutes to remove trapped air and poured at 500 μm thickness onto a micropattemed cyclic olefin copolymer (COC) mold consisting of parallel grooves with 2 μm period, 1 μm groove width and 0.6 μm groove depth. Subsequently, the PDMS was briefly degassed for a second time and cured for 4 hours at 60° C. The cured PDMS patches were separated from the mold with tweezers and cut into squares of 1 cm² with a scalpel. Blank patches were similarly created by pouring PDMS onto flat COC substrates for comparison purposes. Subsequently, all patches were left in ethanol overnight to dissolve any uncrosslinked material. The patches were then treated with oxygen plasma to increase the hydrophilicity of the surface. A process time of 120 seconds at 10 W was chosen after testing a range of intervals from 30 to 150 seconds as the one yielding the lowest contact angle (20.2±0.5°). FIG. 9 shows the testing so the water static contact angle measured on the active PDMS patch surface upon different plasma treatments, the contact angle of untreated PDMS patches is compared with the contact angle of patches treated with low power (10 W) plasma for 30, 60, 90, 120, and 150 seconds and with the contact angle of gelatin coated PDMS. The stiffness of the resulting patches was measured by uniaxial testing and their Young's modulus was calculated to be 1.53±0.057 MPa. As individual fibroblasts can produce traction forces in the 10-100 nN range, it is reasonable to assume that the deformation of topographic features on the surface during wound healing is negligible.

Constructs and Transfection:

In the experiments where focal adhesions and actin filaments visualisation was required, cells were transfected using a Neon Transfection System (Invitrogen, USA). LifeAct-EGFP (Green Fluorescent Protein) and Vinculin-FP635 (Far Red Fluorescent Protein) constructs were used.

Antibodies:

Mouse monoclonal [A17] anti-fibronectin antibody (ab26245) was purchased from Abeam (USA) and secondary goat anti-mouse IgG-FITC antibody was purchased from Sigma Aldrich (USA).

Cell Culture:

Human dermal foreskin fibroblasts (HDF) were supplied by the Tissue Biology Research Unit (Department of Surgery, University Children's Hospital Zurich, CH) and obtained according to the principles of the Declaration of Helsinki. Human juvenile foreskin samples were digested overnight at 4° C. in dispase (0.5 mg/ml, Roche, CH) in Hank's buffered salt solution (HBSS without Ca²⁺ and Mg²⁺, Invitrogen) containing 5 μg/ml gentamycin. This allowed subsequent separation of epidermis and dermis using forceps. To establish primary dermal fibroblast cultures, the dermis was dissociated into single-cell suspensions using HBSS containing collagenase III (1 mg/ml, Worthington Biochem., USA) and dispase (0.5 mg/ml, Roche) at 37° C. for 1 hour. Finally, the cells were cultured in RPMI-1640 medium supplemented with 10% v/v Foetal Bovine Serum, 2 mM L-Glutamine, 100 U/ml Penicillin and 100 g/ml Streptomycin (all from Sigma Aldrich) and maintained at 37° C. and 5% CO2. In all reported experiments, cells with less than five passages in vitro were used.

Both the PDMS patches and the tissue culture plates were coated with gelatin as follows: 1.5% gelatin (Merck, USA) in water was added to the samples and let to adsorb for 1 hour at room temperature (RT). Subsequently, the gelatin was cross-linked by incubating with 2% glutaraldehyde (Sigma Aldrich) in water for 15 minutes at RT. After a sterilization step with 70% Ethanol in PBS (Sigma Aldrich), the substrates were washed 5 times with PBS and left overnight at RT in 20 mM Glycine (Sigma Aldrich) in PBS to neutralize the glutaraldehyde. Finally, the PDMS patches were washed 5 times with PBS and stored at 4° C. until use.

To generate confluent monolayers, the cells were seeded on an unstructured basal support (i.e. 10 cm² tissue culture wells in 6-well plates or in a custom built frame with six glass bottomed dishes) at a density of 5×10 cells/cm² and cultured for 2 days. In order to facilitate automatic wound coverage segmentation by microscopy, the confluent monolayers were treated for 30 minutes with 5-chloromethylfluorescein diacetate (CellTracker™ Green CMFDA, Invitrogen) at 1.5 μg/ml. This concentration was calibrated as the lowest to still ensure good image quality along the entire wound healing experiment. After staining, the monolayers were washed with PBS and a straight wound was induced mechanically with a pipette tip. The average initial wound size in the reported experiments was 539±10 μm. Subsequently, the cultures were gently washed twice with complete medium to remove cell debris and the gelatin-coated PDMS patches were applied on top of the cultures and stabilized with transparent glass weights. In all experiments. 3 patterned patches and 3 blank patches were imaged in parallel.

Decellularization and Immunostaining:

In order to image the fibronectin fibres deposited by the cells on the basal support after complete wound coverage, the patches were gently removed and the cultures were decellularized for subsequent fibronectin staining. For this, the cultures were washed with PBS and the cell membranes were lysed by adding a solution containing 0.5% (v/v) Triton X-100 (Sigma Aldrich) and 20 mM NH₄OH in PBS. The specimens were then left overnight in PBS at 4° C. to fully dissolve cellular debris. The following day, the PBS was gently aspirated and the deposited fibronectin was stained as follows: The specimens were incubated first for 1 hour in blocking buffer (5% BSA in PBS) and then overnight (at 4° C.) with primary antibody. After washing 3 times (1 hour each) with blocking buffer, the specimens were incubated with the secondary antibody for 1 hour at RT. The samples were finally washed five times with PBS and immediately imaged.

Wide-Field Microscopy:

Cell imaging was performed using an inverted Nikon-Ti wide-field microscope (Nikon, Japan) equipped with an Orca R-2 CCD camera (Hamamatsu Photonics, Japan). After patch mounting, the plates were placed under the microscope in an incubated chamber (Life Imaging Services, CH), where temperature, CO₂ concentration, and humidity were maintained at 37° C., 5%, and 95% respectively. Images were collected with a 20×, 0.45 NA long-distance objective (Plan Fluor, Nikon). Nine adjacent non-overlapping fields were recorded in parallel for each sample. This allowed for parallel time-lapse imaging of wound healing with an effective field of view of 1290×983 μm. Parallel movies were acquired with time resolution of 1 hour and a total duration of 31 hours or more. At each time of measure a transmission and a fluorescent image were acquired using a differential interference contrast (DIC) and a FITC filter set, respectively. Focal drift during the experiment was avoided using the microscope's PFS autofocus system.

Fluorescent images of newly deposited fibronectin were obtained with a 40×, 1.30 NA oil immersion objective (PlanFluor, Nikon) using a FITC filter. For each well, the exact location of the original wound was automatically re-located using the motorized stage. Z-stacks (sampling distance of 300 nm) were collected in three different locations within the wound and in one control location away from the wound.

Fluorescent images of HDF expressing LifeAct-EGFP and Vinculin-FP635 were collected with a 60×, 1.2 NA water immersion objective (PlanApo, Nikon) using a FITC and a TRITC filter, respectively.

Image Analysis:

Wound healing movies were analyzed using ImageJ (National Institutes of Health, USA) with the following protocol: The fluorescent channel was contrast-enhanced and thresholded to provide a black and white image. The thresholded images were then despeckled to reduce noise. The wound boundaries were automatically detected in the first image using the “tracing” tool of ImageJ and were saved in order to quantify wound healing dynamics. For each frame of the time-lapse, the cell coverage within the original wound region was measured, thus providing a quantification of wound coverage (in μm²) at each time of measure.

In order to quantify individual cell migration and orientation, the thresholded images were further examined and, where necessary, overlapping cell profiles were manually separated inside ImageJ. Individual cells were detected using the “analyze particles” tool of ImageJ and the cell orientation was measured by using the “fit ellipse” tool. The resulting values were normalized to the initial wound orientation: An angle of 00 indicates an orientation parallel to the wound, whereas 90° indicates orientation perpendicular to the wound. Cell migration tracks were extracted using the ‘particle tracker’ plug-in of the software Imaris (Bitplane, CH). In particular, only migratory tracks continuously detected for a minimum of 15 hours were extracted and the corresponding length, average velocity, overall displacement and travelled paths were automatically calculated.

In order to measure the orientation of fibronectin fibers, the corresponding z-stacks were loaded into ImageJ and their average projections were obtained. Subsequently, fast Fourier transform (FFT) was applied (by using the “FFT” tool of ImageJ) to identify the direction of maximum spatial frequency of intensity variations (the major axis of the resulting ellipse) and, therefore, reveal the direction perpendicular to the principal orientation of the fibers. Thus, the principal orientation of the fibers, relative to the wound, was extracted from the FFT image as parallel to the minor axis of the resulting ellipse.

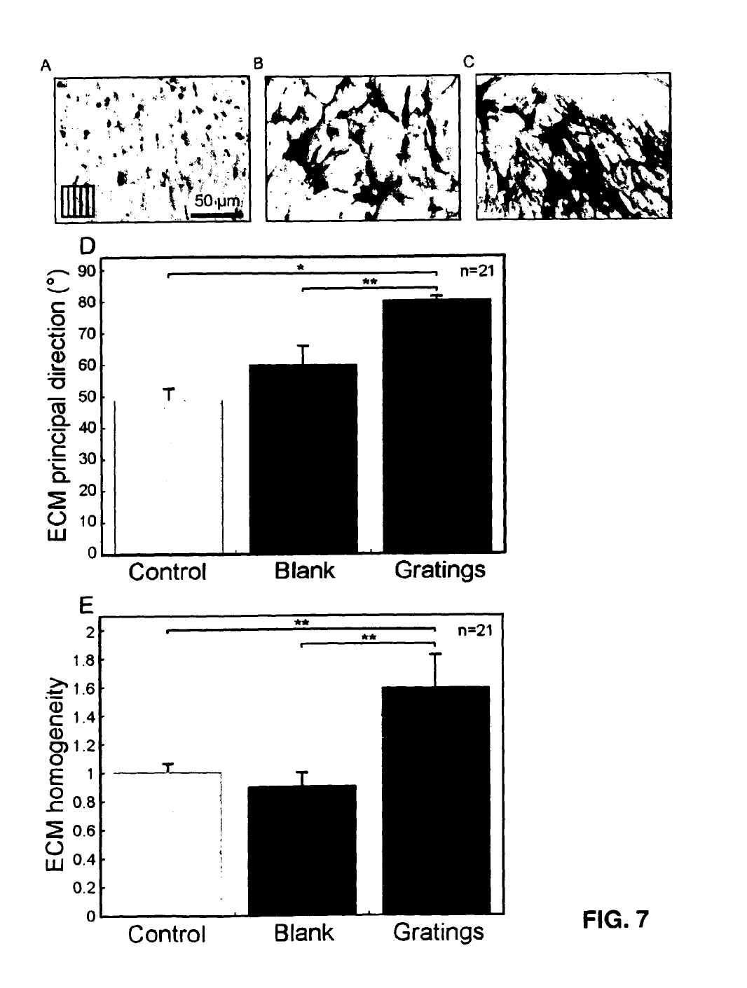

To calculate the fibronectin matrix homogeneity, the standard deviation of the pixel intensity was measured in each average projection image using the “Measure” tool of ImageJ. ECM homogeneity was defined as the inverse of this standard deviation. FIG. 7 shows the results, i.e. shows the architecture of cell-deposited fibronectin, wherein the apical interaction with perpendicular gratings influences the deposition of fibronectin by migrating fibroblasts is shown. In (A) an inverted fluorescent image of fibronectin fibers deposited on the basal support by cells migrating in the wound area under the perpendicular gratings, or (B) blank patch are shown, in (C) randomly oriented fibronectin deposited by cells in unwounded regions, in (D) orientation of fibronectin fibers deposited in the wound region under perpendicular gratings (gray), blank patch (black) or in an unwounded region (light gray), an orientation of 90° indicates alignment perpendicular to the wound (gratings vs. blank: p=0.02, gratings vs. control p=0.02), in (E) homogeneity of the fibronectin matrix (gratings vs. blank: p=0.005, gratings vs. control p=0.004). Coloring as in (D).

For the measurement of focal adhesion number and size, fluorescent images were loaded in ImageJ and individual focal adhesions were manually counted by using the “cell counter” plug-in. The profile of individual focal adhesions was manually drawn using the “Freehand selection” tool. A value for the focal adhesion size (in 1 μm²) was obtained using the “Measurement” tool.

Statistical Analysis:

Statistical analysis was performed in MATLAB (The MathWorks, USA). The differences in wound healing, cell migration, fibronectin orientation and focal adhesion number and size between cultures under the patterned and blank patches were examined by using the Mann-Whitney-Wilcoxon rank sum test (a=0.05). Comparison of cell orientation and cell migration orientation were performed by chi-squared independence test, a=0.05. All quantitative measurements reported are expressed as average values±the standard error of the mean. The total number of events counted is displayed in the upper right corner of the graphs. When not explicitly displayed, the confidence interval for the statistical tests is reported with one, two and three asterisks as p<0.05, p<0.01 and p<0.001, respectively.

Results

Fibroblast Wound Healing:

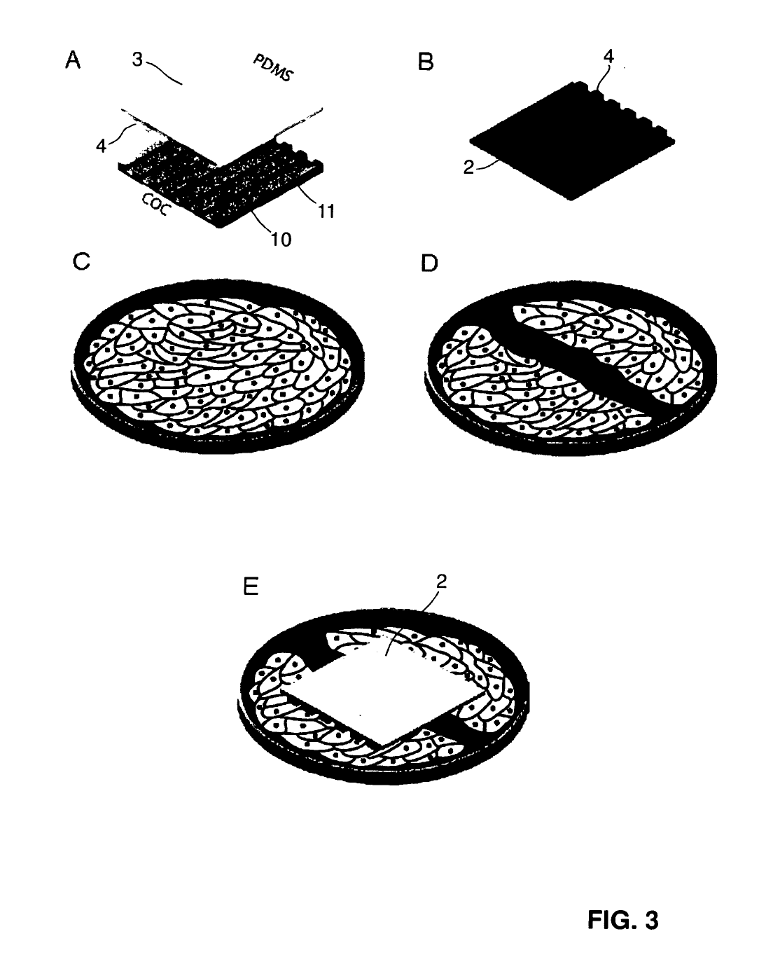

In order to test the effect of the PDMS patches on cell migration in vitro, freshly isolated human dermal fibroblasts (HDF) were grown to confluence on a gelatin-coated basal support. A wound was then mechanically induced in the monolayer with a pipette tip, and the active gelatin-coated surface of the PDMS patches was applied apically to the culture as depicted in FIG. 3. FIG. 3 shows an illustration of the experimental setup, wherein in (A) A PDMS active surface element or patch is generated by soft lithography, in (B) the PDMS patch is plasma-treated to obtain a hydrophilic surface for supporting the gelatin coating (green), in (C) a confluent layer of primary human dermal fibroblasts (HDF) is obtained by culturing cells on a gelatin coated basal support (a Petri dish) in (D) the monolayer is mechanically wounded and in (E) the active surface of the patch is applied over the wound.

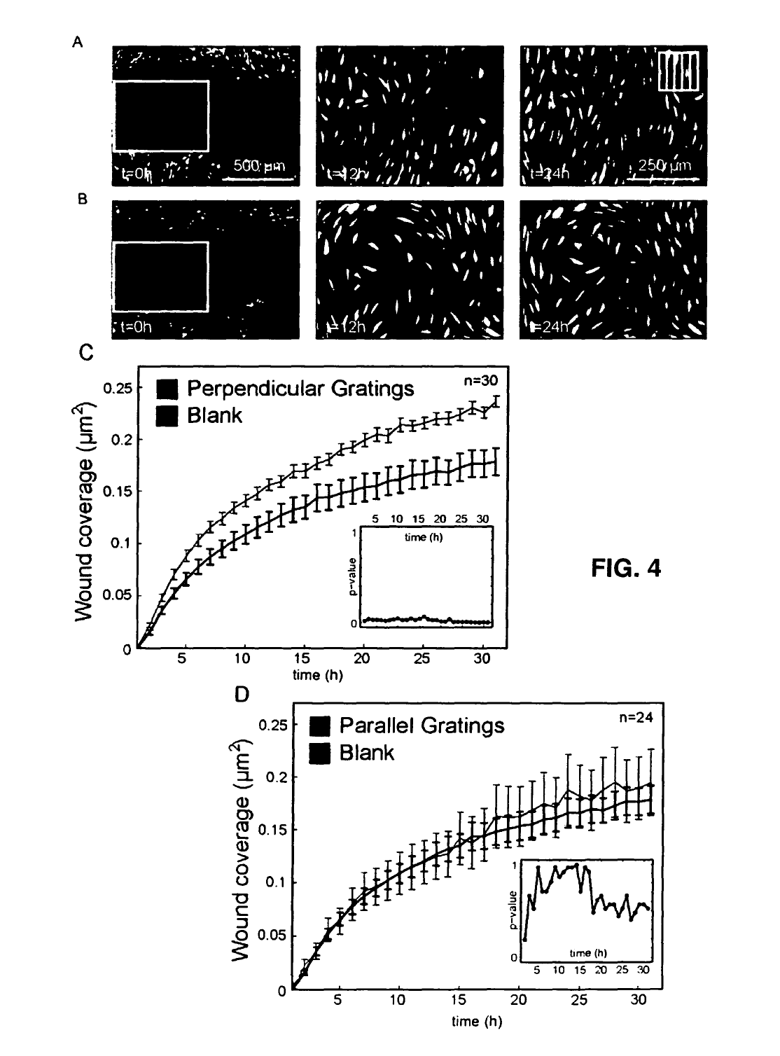

FIG. 4 shows the dynamics of HDF wound healing under perpendicularly oriented gratings (FIG. 4A) or under a blank patch (FIG. 4B). Shortly after wounding (t=0 h, FIGS. 4A and 4B) the cells started migrating into the wound from the edge regions. Cell coverage of the wound was evident already after 12 hours under the perpendicular gratings, and cells could re-establish a confluent monolayer after 24 hours (FIG. 4A). Importantly, in the same experimental conditions, wound healing under a blank patch proceeded less efficiently as large uncovered regions were present at 12 hours and low confluence was still evident at 24 hours after wounding (FIG. 4B). In order to quantify the difference in wound healing dynamics under perpendicular gratings or a blank patch, the cell-coverage in the wound area was measured over the entire wound healing process. The graph in FIG. 4C depicts the wound coverage over time and shows, for the perpendicular gratings and the blank patch conditions, a two-phase behaviour: Between 0 and 10 hours after wounding, the wound coverage grew rapidly, while at a later stage (between 10 and 30 hours after wounding, FIG. 4C) the coverage tended to a plateau. Importantly, the coverage was significantly higher under perpendicular gratings at the end of the initial phase, and this difference was maintained during the later slow phase (FIG. 4C). These results suggest that the cellular processes supporting wound healing were both faster and more efficient under the topographically modified patch. When the gratings were oriented parallel to the wound, the wound coverage dynamics were similar to those obtained under a blank patch (FIG. 4D), indicating that the healing effect depends on the relative orientation between the gratings and the wound.

Apical Guidance During Wound Healing:

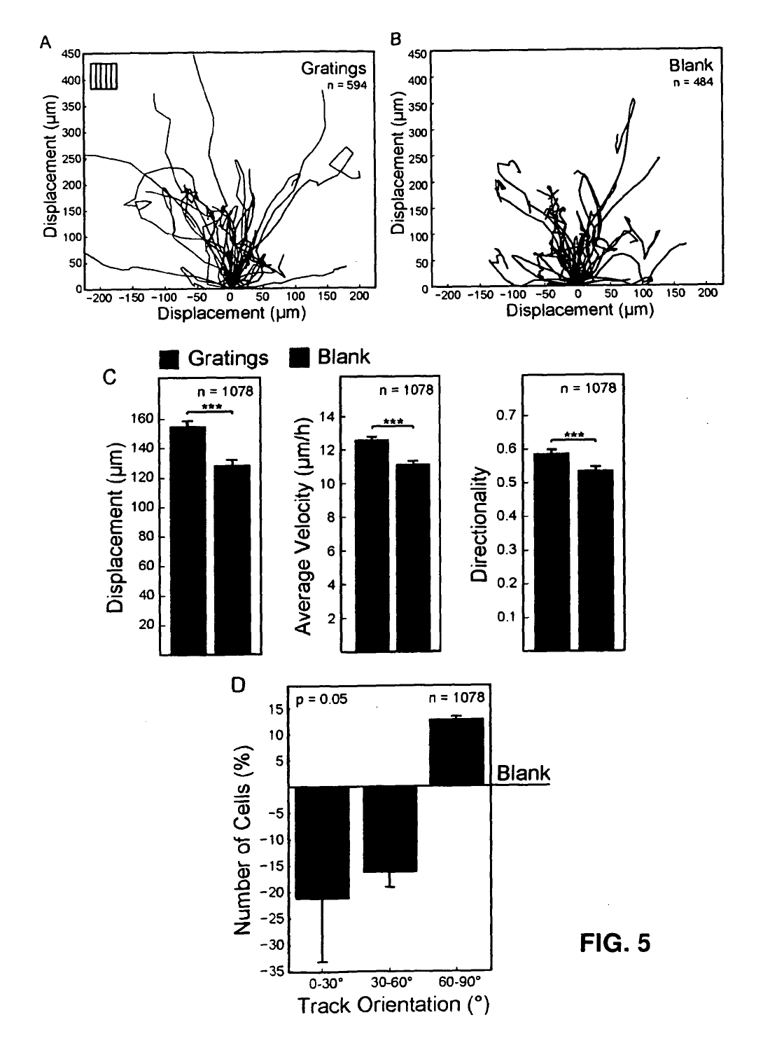

In order to evaluate whether the measured effect of the perpendicular gratings (FIG. 4) is based on a guidance mechanism, individual tracks of migrating cells were extracted from wound healing movies. The analysis of tracks obtained under perpendicular gratings (FIG. 5A) and under a blank patch (FIG. 5B) revealed that cells in contact with the topographically modified surface migrated over longer distances and in straighter paths, thereby penetrating deeper into the wound area. The average cell displacement from the original position to the final position upon wound healing was 21% higher for cells migrating under the perpendicular gratings (FIG. 5C) compared to cells migrating under the black patch. Longer migration tracks resulted from faster movement (the average migration velocity was 13% higher under the perpendicular patch) and improved directionality (the ratio of total distance traveled over total displacement was in average 10% lower under the perpendicular patch). Importantly, these activities were translated into faster wound coverage by better track orientation as shown by a significant increase (13%) in the percentage of tracks aligned within 60 to 90 degrees toward the wound (FIG. 5D).

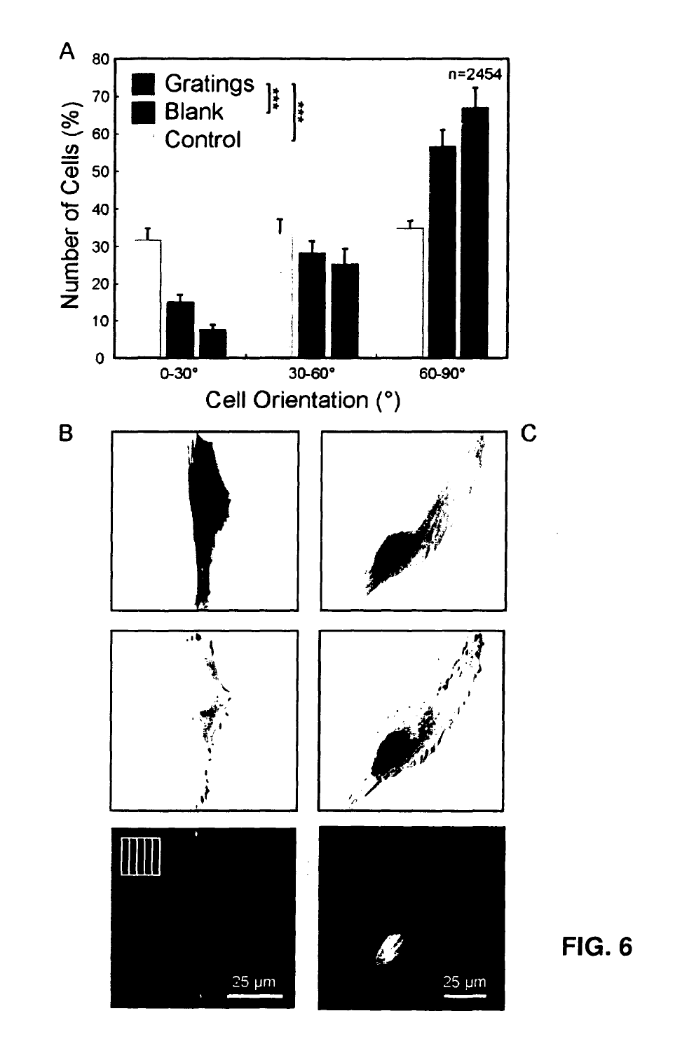

Analysis of individual cell polarization (FIG. 6) supports the results of the migration track study: Cells under the perpendicular gratings were better aligned toward the direction of the gratings and thus perpendicular to the wound. In particular 18% more cells aligned within 60 to 90 degrees relative to the direction of the wound. The global distribution and orientation of focal adhesions and microfilaments in cells migrating under perpendicular gratings (FIG. 6B) or under a blank patch (FIG. 6C) further reveal an improved cell orientation which correlates with adhesion alignment, supporting the generation of actin stress fibers along the main cell axis. Altogether, these results suggest that perpendicular gratings contribute to orient the migration of underlying fibroblasts by reinforcing cell polarization along the topography.

Apical Guidance Results in Aligned ECM Fibres Deposition:

The architecture of fibronectin fibres newly deposited by migrating fibroblasts into the wound region strongly influences the transition to wound resolution or scaring in vivo. In order to test whether the guidance effect induced by the perpendicular gratings (FIGS. 4-6) influenced ECM deposition by migrating HDF, the global architecture of fibrillar fibronectin deposited into the wound area was visualized after complete healing (FIG. 7). Fibronectin fibres deposited (or remodelled) on the basal support (FIG. 3) by fibroblasts penetrating into the wound under perpendicular gratings were homogeneously distributed and showed a basketweave organization with preferential alignment in the direction of the gratings (FIG. 7A). In sharp contrast, the matrix deposited by cell migrating under a blank patch was less organized and showed regions of varying fibronectin density (FIG. 7B) similar to those found in unwounded regions of the monolayer (FIG. 7C). Fourier analysis of global matrix alignment (FIG. 7D) confirmed that fibroblasts migrating under perpendicular gratings aligned fibronectin fibres perpendicularly to the wound while the alignment of fibres deposited under a blank patch was significantly worse (59.8°±6.1°). We next quantified the homogeneity of fibronectin deposited upon wound healing. A significantly lower standard deviation of the pixel intensity for wounds healed under the perpendicular patch revealed that the matrix was more homogeneous than under the black patches or in control unwounded regions (FIG. 7E).

In summary, these results demonstrate that the apically applied topography influences the global architecture of the matrix deposited in the wounded region, yielding better overall distribution and orientation of the fibres.

Apical Guidance does not Require the Establishment of New Focal Adhesions:

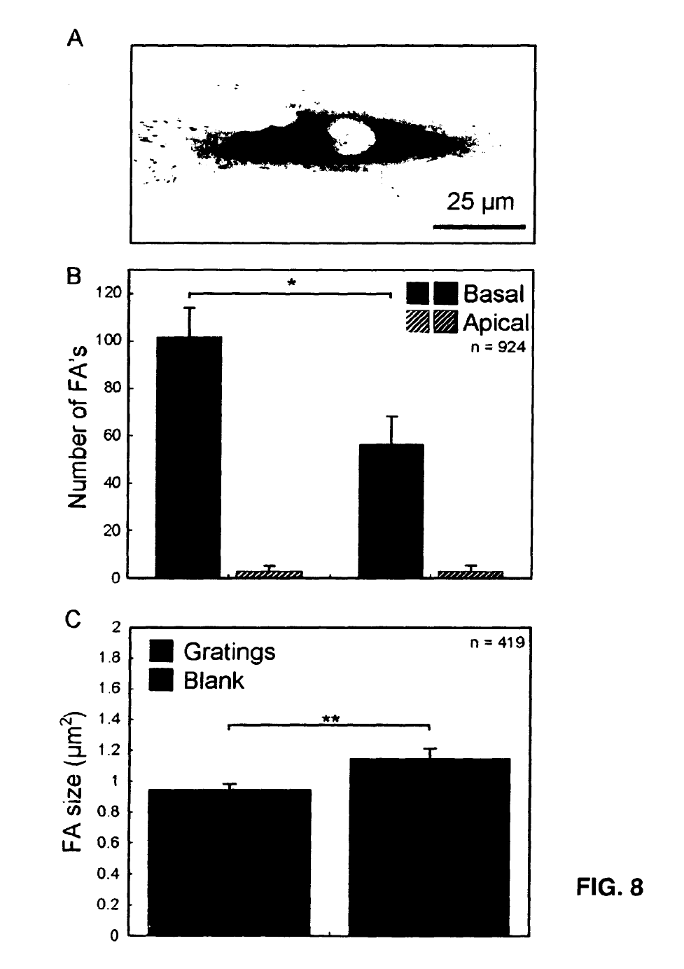

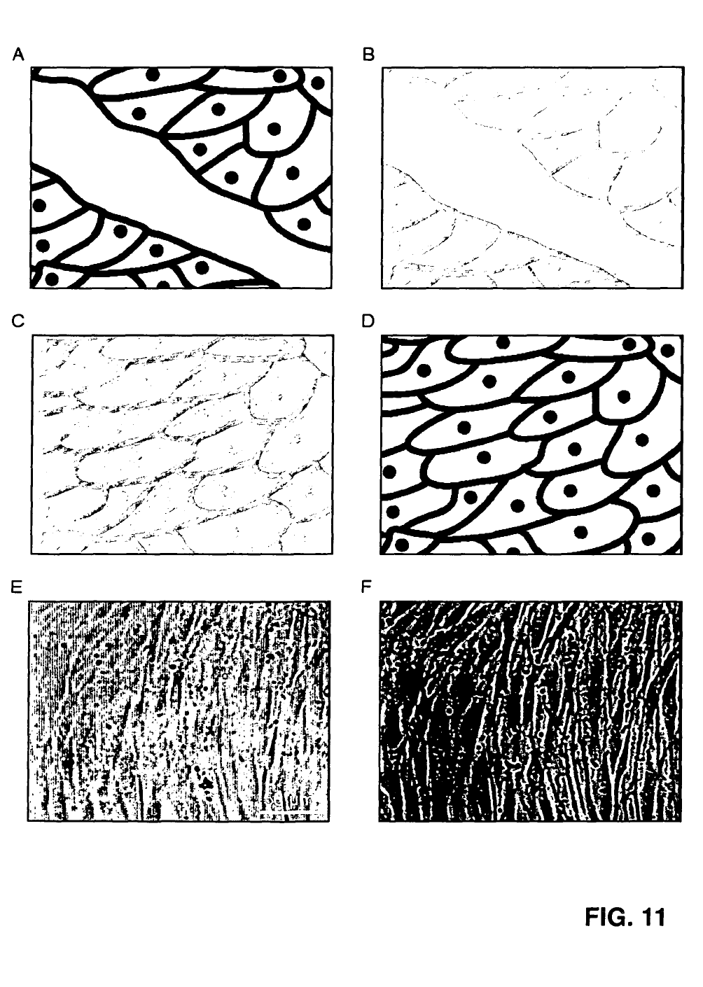

Is the guidance effect induced by the apical application of perpendicular gratings (FIGS. 4-6) required an interaction between topographical features and focal adhesions? The size and location of focal adhesions established by fibroblasts was revealed by the transient expression of vinculin-FP635 (FIG. 8A). Under our experimental conditions, overexpression of vinculin did not affect the migration and polarization of HDF. Fibroblasts established focal adhesions at the interface with the basal support as revealed by punctuate fluorescent signal (FIG. 8A). Importantly, under both experimental conditions, only a minimal number of cells established few, optically resolvable focal adhesions at the interface with the apical patch (FIG. 8B). Indeed, the average number of focal adhesions established by HDF with the basal support was 101±12 for cells migrating under perpendicular gratings and 56±12 for cells under a blank patch, while the average number of adhesions established with the apical patch was in both cases less than 2. This result indicates that the biological interaction with the basal support was significantly stronger than with the apical patch. To confirm this hypothesis, the patches were removed after complete wound healing. FIG. 11 shows the patch removal after complete wound healing, wherein in (A) an illustration of a wounded monolayer before and (B) after patch application is shown, in (C) the healed monolayer before and (D) after patch removal, and in (E) a DIC image of a healed region before and (F) after patch removal. In all cases, the patch removal could be accomplished without damaging the healed monolayer (FIGS. 11E and 11F) or stripping the cells off. FIG. 10 shows patch removal after complete wound healing: (A) Illustration of a wounded monolayer before and (B) after patch application. (C) Illustration of the healed monolayer before and (D) after patch removal. (E) DIC image of a healed region before and (F) after patch removal. Interestingly, the average size (i.e. the maturation stage) of adhesions established by fibroblasts migrating under perpendicular gratings was significantly smaller than the size of adhesions established by cells under a blank patch (0.95±0.04 vs. 1.14±0.07 μm²; FIG. 8C). This result is consistent with an increased migrating phenotype displayed by cells under perpendicular gratings (FIG. 5). Altogether, these data demonstrate that guidance induced by apically applied perpendicular gratings on HDF is not mediated by the interaction between cell-established focal adhesions and the topographical features on the surface. Instead, the observed effect has to be ascribed to a novel, focal adhesion-independent mechanism.

LIST OF REFERENCE SIGNS

| | | | |
|---|---|---|---|
| 1 | healing patch | a | healing patch length |
| 2 | patterned active surface | b | healing patch width |
| | element | c | active surface element length |
| 3 | backside of 2 | d | active surface element width |
| 4 | frontside of 2, topographic | e | ridge width |
| | surface | f | groove width |
| 5 | ridge | α | pattern angle |
| 6 | groove | p | pattern period |
| 7 | central mirror plane of the | h | pattern height |
| | ridge | l | pattern length along running |
| 8 | central mirror plane of the | | direction of grooves/ridges |
| | groove | | |
| 9 | running direction of the | | |
| | pattern | | |
| 10 | mold element | | |
| 11 | complementary structure on | | |
| | 10 | | |

1. Active surface element (2) for improved healing of cell layer lesions comprising at least one topographically structured surface on a substrate, with a pattern comprising alternating ridges (5) and grooves (6) with a pattern period (p) and extending along a pattern length (l), wherein
the pattern period (p) is smaller than 10 μm and wherein
the pattern length (l) is larger than 1 mm and/or the pattern length (l) is larger than 20 times the period (p) of the structure.

2. The surface element according to claim 1, wherein the width of the ridges (5) and/or of the grooves (6) is in the range of 1-9μ, wherein preferably the width of the ridges (5) is in the range of 1-5μ and the width of the grooves (6) is in the range of 1-5μ, preferably both widths being essentially equal.

3. The surface element according to any of the preceding claims, wherein the ridges (5) have a height (h) of at least 0.4 μm, preferably in the range of 0.5-5 μm or in the range of 0.5-2 μm, more preferably in the range of 1-2 μm.

4. The surface element according to any of the preceding claims, wherein the sidewalls of the grooves (6) and a bottom wall of the grooves (6) enclose a pattern angle (α) in the range of 85-120°, wherein preferably the pattern angle (α) is around 90°.

5. The surface element according to any of the preceding claims, wherein the ridges (5) and/or the grooves (6) are mirror symmetric with respect to a respective central plane (7, 8, respectively) parallel to the running direction (9).

6. The surface element according to any of the preceding claims, wherein it comprises a substrate based on or consisting of a biocompatible polymeric material, preferably selected from the group consisting of: polycaprolactone, polyethylene glycol, polylactic acid, polyglycolic acid, polybutyric acid, as well as mixtures, derivatives, hydrogels and copolymers thereof.

7. The surface element according to claim 6, wherein the biocompatible polymeric material as a Young's modulus of at least 100 kPa, preferably in the range of 100 kPa-10 GPa.

8. The surface element according to any of claims 6-7, wherein the surface is coated, uncoated and/or plasma treated.

9. The surface element according to any of claims 6-8, wherein the substrate has an open porosity with pores with a diameter in the range of 1μ-1 mm, preferably in the range of 1μ-2μ.

10. The surface element according to any of the preceding claims further comprising a backing material adhesively attached on the side opposite to the topographically structured surface, wherein said backing material is adapted for supporting the surface element and/or for allowing to adhesively attach the combined structure to the skin of a patient, and wherein preferably the backing material is a multilayer structure including layers for absorption as well as layers for adhesion purposes.

11. The surface element according to claim 10, wherein the backing material is an absorbent backing material, preferably selected from the group consisting of: cotton, viscose, cellulose, silk, or combinations thereof, in woven or nonwoven forms.

12. Method for making a surface element according to any of the preceding claims, wherein a topographically complementary structured mould element is used as a template for a liquid applied or injected substrate material, preferably in a soft lithography process, optionally followed by a cross-linking and/or polymerization step, further optionally followed by a surface treatment step, preferably a plasma treatment step on the topographical surface (4).

13. Bandage, preferably adhesive bandage comprising at least one surface element according to any of the preceding claims 1-11, wherein preferably the orientation of the pattern length (l) of the surface element on the adhesive bandage is arranged such as to lie essentially perpendicular to the corresponding lesion, preferably to a skin lesion, or preferably including a cut in epidermis and/or dermis and/or hypodermis cell layers.

14. Method for wound healing of cell layer lesions, preferably skin cell layer lesions, most preferably lesions in epidermis and/or dermis and/or hypodermis cell layers, comprising the step of applying a surface element according any of the preceding claims 1-11 on the lesion, preferably in a relative orientation such that the orientation of the pattern length (l) is under an acute angle or preferably perpendicular to the main orientation of the lesion, allowing the regeneration of the cell layers, and removal of the surface element or biodegradation of the surface element.
